(19) United States
(12) Patent Application Publication
(10) Pub. No.: US 2012/0225903 A1
(43) Pub. Date: Sep. 6, 2012

(54) Novel 2-Substituted Quinoline Derivatives, And Method For Preparing Same

(76) Inventors: Alain Fournet, Ossages (FR); Bernardin Akagah, Chatenay-Malabry (FR); Bruno Figadere, Saint Cheron (FR); Anh Tuan Lormier, Fresnes (FR)

(21) Appl. No.: 12/993,102

(22) PCT Filed: May 15, 2009
(86) PCT No.: PCT/FR2009/000573

§ 371 Date: Feb. 28, 2011

(30) Foreign Application Priority Data
May 20, 2008 (FR) 0802745

(51) Int. Cl. A61K 31/47; A61K 31/4741; C07D 491/153; A61P 31/18; C07D 215/24; C07D 215/26; A61P 33/02; A61P 31/14; C07D 491/044; C07D 215/14

(52) U.S. Cl. 514/287; 546/89; 514/291; 546/65; 546/168; 514/311; 546/179

(57) ABSTRACT

The invention relates to 2-substituted quinoline derivatives, to a method for preparing same, and to the use thereof for drug production, particularly for treating infections caused by protozoa, such as leishmaniases, trypanosomiases, toxoplasmoses, and/or infections caused by retroviruses such as, for example, HIV or HTLV.

The subject of the invention is novel 2-substituted quinoline derivatives, a process for the preparation thereof and the use thereof for the production of medicaments.

Substituted quinolines of varied structures have been described for their action in the treatment of infections caused by protozoa, such as leishmaniasis, trypanosomiasis, toxoplasmosis, and/or infections caused by retroviruses, for instance HIV or HTLV.

Some molecules among the quinolines, and in particular the 2-substituted quinolines, have shown an advantageous potential in the treatment of these pathological conditions. However, there remains the need for molecules which are even more active and which are capable of being produced on an industrial scale.

Specifically, in order to enable the development of a medicament, a molecule must at the same time exhibit satisfactory in vivo pharmacological activity, low toxicity, and also the possibility of being produced industrially with acceptable costs.

A strategy sometimes used for discovering novel active ingredients consists in subjecting the latter to the action of the biological medium for which they are intended and then in identifying the metabolites formed. Said metabolites often constitute very effective active ingredients. However, this approach has certain drawbacks: in vivo studies in animals pose ethical problems, and the identification of the metabolites in an organism, in sometimes very small amounts, is difficult. In vitro metabolic studies do not come up against the ethical difficulties, but these studies very often result in such small amounts of products that it is difficult to identify said products. The complexity of the reactions owing to the presence of microorganisms in the medium does not make it possible to identify all the compounds which may have a therapeutic interest, since some are reconverted directly without it being possible to identify them.

Several authors have proposed using metalloporphyrins (MEPs) to mimic reactions produced in mammals by cytochrome P450 monooxygenases, the main enzymes involved in the oxidative metabolism of medicaments (WO01/10797; Chorgade M. S. et al., Pure Appli. Chem., 1996, 68, 753; Mansuy D. et al., Eur. J; Soc. Biochem. 1989, 184, 267361; Komur M. et al., J. Chem. Soc. Chem. Perkin Trans. I, 1996, 18, 2309; Meunier B. Chem. Rev., 1992, 92, 1411). Specifically, some MEPs, in the presence of oxygen donors, form an oxometallic species which mimics the cytochrome enzyme reaction site (Chauhan S. M. S. et al., Chem. Pharm. Bull., 2003, 51, 1345).

During previous studies, various 2-substituted quinoline metabolites have been identified. However, the method used, an in vitro study by means of liver microsomes, hepatocytes and recombinant enzymes, did not make it possible to isolate any molecules. Only an identification of certain species by LC/MS was possible (Desrivot J. et al., Toxicology, 2007, 235, 27). This study did not therefore make it possible to evaluate the therapeutic potential of the compounds formed.

Various tests for producing 2-substituted quinoline metabolites, which are unpublished, did not make it possible to achieve the expected compounds.

There therefore remained the need for a method for producing 2-substituted quinoline metabolites and also other derivatives of these molecules, so as to enable them to be studied from the point of view of their pharmacology and their toxicity.

A first subject of the invention is a process for producing 2-substituted quinoline-derived molecules, these molecules having in common the fact that they are 2-substituted quinoline metabolites or metabolite analogs.

This process is characterized in that it comprises at least one step during which a molecule corresponding to general formula (I):

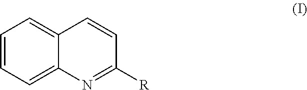

in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxy-carbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group;
   is reacted with an MEP, in the presence of an oxidizing agent, in a solvent.

Preferably, R is chosen from:
a C₁ to C₈ alkyl group, a C₂ to C₈ alkenyl group or a C₂ to C₈ alkynyl group;
a C₁ to C₈ alkyl or C₂ to C₇ alkenyl group bearing at least one substituent chosen from oxygen, halogens and hydroxyl, formyl, carboxyl, cyano (CN) and amine (NH₂) groups;
a C₂ to C₇ alkynyl group bearing at least one substituent chosen from oxygen, halogens and hydroxyl, formyl and carboxyl groups.

Even more advantageously, R is chosen from:

a C₂-C₅ alkyl or alkenyl group optionally bearing one or more functions chosen from: —OH and CN.

The oxidizing agent is advantageously chosen from: H₂O₂, sodium hypochlorite, iodosylbenzene, chloroperbenzoic acid, tert-butyl hydroperoxide and 2,6-dichloropyridine N-oxide.

Preferably, the oxidizing agent is hydrogen peroxide H₂O₂, which is advantageously used at a concentration of 30% to 45% in solution in water.

The reaction is carried out in a solvent or a mixture of solvents. The solvent is advantageously chosen from: water, acetonitrile, dichloromethane, chloroform, and also mixtures thereof, and advantageously a mixture of acetonitrile and dichloromethane.

Advantageously, the reaction is carried out in the presence of H₂O₂ and of an imidazole acting as cocatalyst, in a solvent or a mixture of solvents composed of dichloromethane and acetonitrile. Other cocatalysts can also be used in place of imidazole in order to make the reaction more efficient. Mention may be made, for example, of: pyridine, histidine, triethylamine, 4-methylpyridine, 2,4,6-trimethyl-pyridine, N,N,N′,N′-tetramethylethylenediamine (TMDEA), N-methylmorpholine and 1,8-diazabicyclo[5.4.0]undec-7-ene (DBU).

The process of the invention makes it possible to obtain molecules of formulae (II) (molecules (IIa), (IIb) and (IIc)) and (III) below:

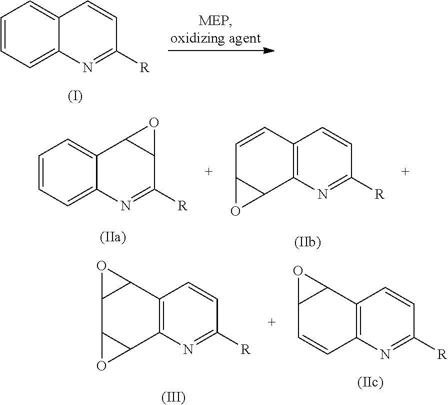

in which R has the same definition as in formula (I).

In addition, in the particular case where R comprises a CH₂ group in the alpha-position with respect to the quinoline ring, the process of the invention makes it possible to carry out the oxidation of the carbon atom in the alpha-position and to convert the molecules of formula (Ia) to molecules of formula (IV) as illustrated below:

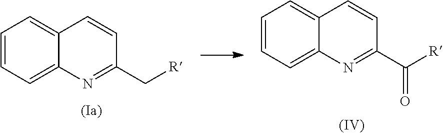

In this case, starting from a product (Ia), five molecules are obtained: (IIa), (IIb), (IIc), (III) and (IV).

In this case, R′ represents a group chosen from:
a C₁ to C₁₄ alkyl group, a C₂ to C₁₄ alkenyl group or a C₂ to C₁₄ alkynyl group;
a C₁ to C₁₄ alkyl or C₂ to C₆ alkenyl group bearing at least one substituent chosen from oxygen, halogens and the following groups: hydroxyl, formyl, carboxyl, C₇ to C₁₃ aryloxycarbonyl, C₂ to C₈ alkyloxy-carbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₆ alkynyl group bearing at least one substituent chosen from oxygen, halogens and the following groups: hydroxyl, formyl, carboxyl, C₇ to Cₙ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl; or else a C₂ to C₁₄ alkenyl or alkynyl group substituted with at least one C₁ to C₇ trialkylsilyl group.

Preferably, R′ is chosen from:
a C₁ to C₇ alkyl group, a C₂ to C₇ alkenyl group or a C₂ to C₇ alkynyl group;
a C₁ to C₇ alkyl or C₂ to C₆ alkenyl group bearing at least one substituent chosen from oxygen, halogens and hydroxyl, formyl, carboxyl, cyano (CN) and amine (NH₂) groups;
a C₂ to C₆ alkynyl group bearing at least one substituent chosen from oxygen, halogens and hydroxyl, formyl and carboxyl groups.

Advantageously, for carrying out this reaction, R′ is chosen from the following groups: H, C₁-C₄ alkyls and C₂-C₄ alkenyls, optionally bearing one or more functions chosen from: —OH and CN.

The molecules thus obtained are done so with yields which are much higher than those that could be obtained by means of the biochemical processes of the prior art. In addition, these processes can be easily extrapolated to the industrial scale. These molecules can thus be easily separated by means of methods well known to those skilled in the art, such as liquid-liquid extraction and chromatography. They can therefore be obtained in isolated form.

The molecules of formulae (I) and (Ia) were prepared as described in the prior art WO 93/07125.

The MEPs that can be used can be defined by the formula below:

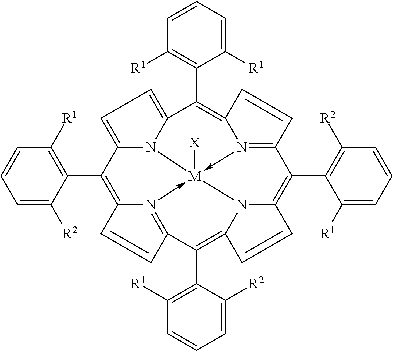

in which M represents a metal atom chosen from: Mn, Ni, Fe, Co, Mo and Cu;
X represents a halogen atom: Cl, Br or I, or an acetate group;
R¹ and R², which may be identical or different, are chosen from halogen atoms: F, Cl, Br or I, and methyl and methoxy groups.

Among the MEPs, some are known (Lindsey J. S. et al., tetrahedron Lett., 1986, 27, 4969; Ram Singh and Geetanjali, J. Braz. Chem. Soc.), in particular the following two MEPs are preferably used for implementing the invention:

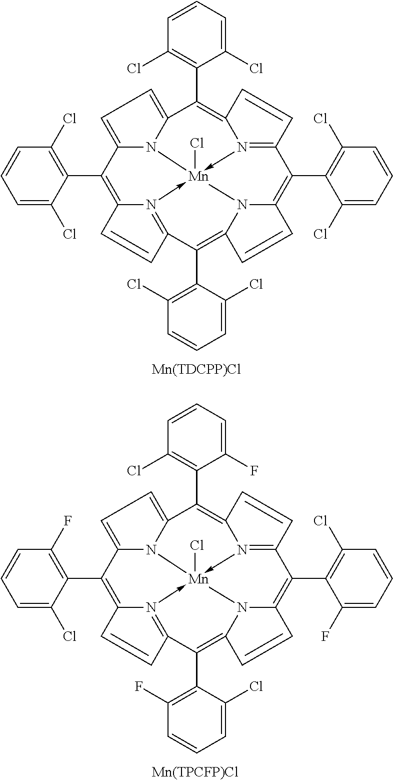

However, use may also be made of a metalloporphyrin chosen from: Fe(TDCPP)Cl, Fe(TPP)Cl, Fe(TPCFP)Cl and Mn(TPP)Cl.
TPP=tetra phenyl porphyrin.
TPCFP=tetra phenyl chloro fluoro porphyrin.
TDCPP=tetra di chloro phenyl porphyrin.

The molecules of formula (IIa) in which R has the same definition as in formula (I) above, with the exclusion of the case where R=n-propyl, are novel and constitute another subject of the invention.

The molecules of formula (IIb) in which R has the same definition as in formula (I) above, with the exclusion of the cases where R═H or R=n-propyl, are novel and constitute another subject of the invention.

The molecules of formula (IIc) in which R has the same definition as in formula (I) above, with the exclusion of the cases where R═H or R=n-propyl, are novel and constitute another subject of the invention.

The molecules of formula (III) in which R has the same definition as in formula (I) are novel and constitute another subject of the invention.

The molecules of formula (IV) in which R′ has the same definition as in formula (Ia) above, with the exclusion of the cases where R′ represents a group chosen from the list below:

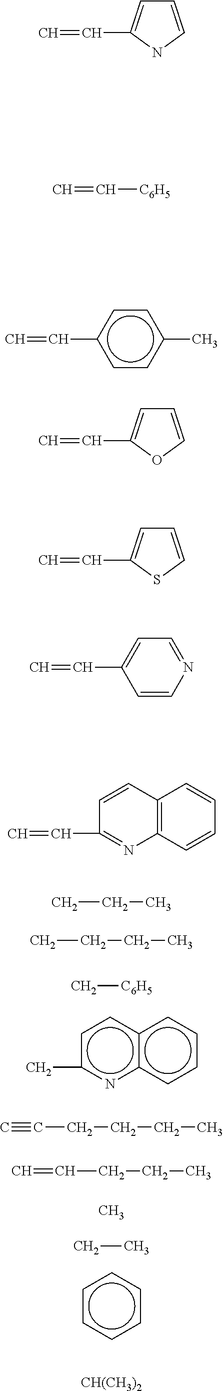

are novel and constitute another subject of the invention.

In addition, the molecules of formula (III) can be subjected to a treatment of hydrolysis in an acid medium which makes it possible to obtain the molecules of formula (V) according to the scheme below:

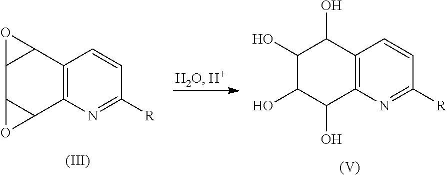

A subject of the invention is also a process for preparing the compounds of formula (V) in which R has the same definition as formula (I). A subject of the invention is also the compounds of formula (V).

In addition, the molecules of formula (V) can be subjected to an oxidation treatment which makes it possible to obtain the molecules of formulae (VI) and (VII) according to the scheme below:

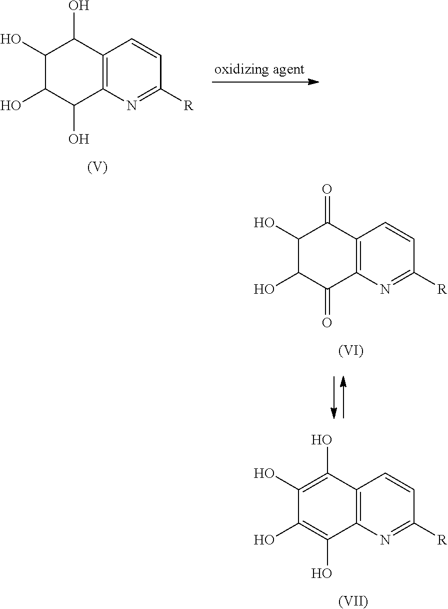

A subject of the invention is also a process for preparing the compounds of formulae (VI) and (VII) in which R has the same definition as for formula (I). A subject of the invention is also the compounds of formulae (VI) and (VII), in tautomeric equilibrium.

In particular, the process of the invention was applied to the n-propyl-2-quinoline 2a so as to give the products 3a, 4a, 5a below:

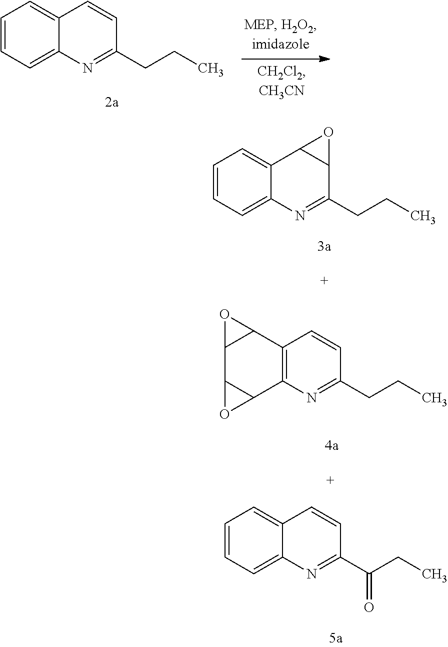

It was also applied to the 2-(3-cyano-n-2-propenyl)-quinoline 2b, so as to give the products 3b, 3c, 3d, 4b and 4c below:

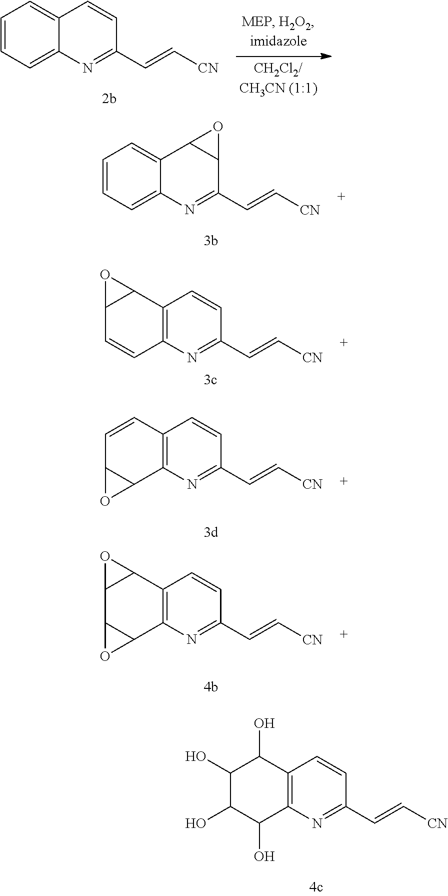

In both cases, the process was applied with MEP=Mn(TDCPP)Cl and MEP=Mn(TPCFP)Cl.

The oxidation of the product 4a was continued so as to give the products 6a, 7a and 8a according to the scheme below:

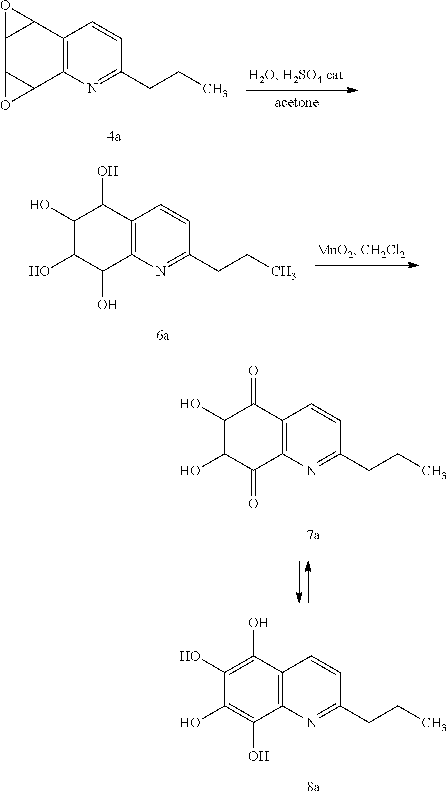

A subject of the invention is also any pharmaceutical composition comprising a product of formula (IIa), (IIb), (IIc), (III), (IV), (V) or (VI), as defined above, and a pharmaceutical carrier, in particular with one or more inert, nontoxic excipients suitable for the pathological condition, for the population to be treated and for the climatic conditions. A subject of the invention is in particular any pharmaceutical composition comprising a product of formula 3b, 3c, 3d, 4a, 4b, 4c, 6a, 7a or 8a and a pharmaceutical carrier.

Among the pharmaceutical compositions of the invention, mention may be made of those which allow oral, parenteral or nasal administration, tablets (plain or sugar-coated), sublingual tablets, gel capsules, lozenges, suppositories, creams, ointments, injectable preparations, oral suspensions, etc.

The dosage is adjusted according to: the pathological condition to be treated, the severity of the condition, the age and weight of the patient, and the route of administration. It can range from 0.01 to 50 mg per day in one or more intakes.

A subject of the invention is also a medicament comprising a molecule of formula (IIa), (IIb), (IIc), (III), (IV), (V) or (VI) as defined above, for use in the prevention or treatment of a disease selected from infections caused by protozoa, such as leishmaniasis, trypanosomiasis, toxoplasmosis, and/or infections caused by retroviruses, for instance HIV or HTLV.

### EXPERIMENTAL SECTION

### Examples

### Example 1

### Catalytic Oxidation of 2a (1 g) in the Presence of Mn(TDCPP)Cl or of Mn(TDCFP)Cl

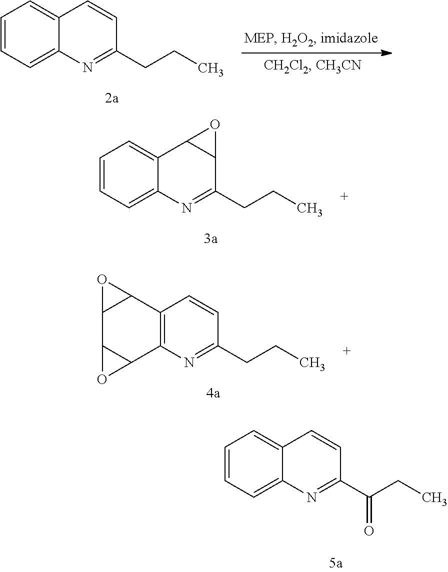

Firstly, a solution composed of 1 g (5.88 mmol) of n-propyl-2-quinoline 2a, 40 mg (0.58 mmol) of imidazole and 133 mg of Mn(TPCFP)Cl in a mixture of 20 ml of CH₂Cl₂/acetonitrile (1/1, v/v) is prepared. Another solution containing 200 mg (2.9 mmol) of imidazole and 3 ml of 35% H₂O₂ (200 equivalents) in 26.5 ml of acetonitrile is added dropwise to the mixture, with stirring, over 1 h30-2 h. The reaction medium is kept stirring at ambient temperature for 2 hours. The solvent is then evaporated off under reduced pressure and the crude residue is purified by silica gel chromatography. The eluent used for separating the oxidation products is a cyclohexane/ethyl acetate (7/3, v/v) mixture. The isolated products 3a, 4a and 5a are identified by GC-MS, NMR and IR.

3a (140 mg): ¹H (300 MHz, CDCl₃) δ 8.12 (dd, J=8.4, 0.9 Hz, 1H), 7.81 (d, J=7.5 Hz, 1H), 7.68 (t, J=7.8 Hz, 1H), 7.48 (t, J=8.1 Hz, 1H), 4.71 (d, J=5.1 Hz, 1H), 4.14 (d, J=5.1 Hz, 1H), 2.30 (m, 2H), 1.36 (m, 2H), 0.61 (t, J=7.2 Hz, 3H);

¹³C (75 MHz, CDCl₃) δ 202.9, 146.9, 133.9, 130.1, 129.6, 129.3, 124.4, 60.4, 57.3, 42.5, 16.1, 13.2;

EIMS (70 eV) m/z (%) 187 (M, 34), 172 (36), 159 (100), 143 (10), 103 (10), 77 (9);

IR cm⁻¹ 2965, 1725, 1525, 1345, 1260, 1015;

4a (431 mg): ¹H (300 MHz, CDCl₃) δ 7.59 (d, J=7.8 Hz, 1H), 7.08 (d, J=7.8 Hz, 1H), 4.01 (dd, J=5.8, 4.0 Hz, 1H), 3.85 (d, J=4.0 Hz, 1H), 3.67 (d, J=4.0 Hz, 1H), 2.74 (t, J=7.4 Hz, 2H), 1.73 (m, 2H), 0.95 (t, J=7.4 Hz, 3H);

¹³C (75 MHz, CDCl₃) δ 162.8, 151.3, 138.7, 124.5, 122.6, 55.3, 54.4, 53.1, 50.7, 39.9, 22.8, 13.6;

EIMS (70 eV) m/z (%) 203 (M, 37), 202 (39), 188 (18), 175 (100), 159 (12), 146 (45), 117 (H), 77 (12);

IR cm⁻¹ 12960, 2925, 2855, 1725, 1580, 1275;

5a (152 mg): ¹H (300 MHz, CDCl₃) δ 8.25 (d, J=8.4 Hz, 1H), 8.18 (dt, J=8.4, 0.4 Hz, 1H), 8.12 (d, J=8.6 Hz, 1H), 7.86 (dd, J=8.0, 1.0 Hz, 1H), 7.77 (td, J=7.0, 1.6 Hz, 1H), 7.63 (td, J=6.8, 1.2 Hz, 1H), 3.42 (q, J=7.4 Hz, 2H), 1.26 (t, J=4.0 Hz, 3H);

¹³C (75 MHz, CDCl₃) δ 203.0, 153.0, 147.1, 136.7, 130.5, 129.8, 129.5, 128.3, 127.6, 118.1, 30.8, 8.0;

EIMS (70 eV) m/z (%) 185 (M, 17), 157 (54), 129(100), 101 (25), 77 (15);

IR cm⁻¹ 2975, 1690, 1560, 1360, 1115, 935.

### Example 2

### Preparation of the Tetraol Derivative 6a

A few drops of concentrated sulfuric acid are added to a 100 ml round-bottomed flask containing 275 mg (1.35 mmol) of bisepoxide 4a in 34 ml of acetone/H₂O (1/1, v/v), with stirring at ambient temperature. The progression of the reaction is monitored by TLC, and after 24 h, NH₃ is added in order to neutralize the pH of the reaction medium. The acetone is evaporated off under reduced pressure and then the aqueous phase is extracted with ethyl acetate (3×20 ml). The organic phases collected are then dried with MgSO₄, and the resulting product is evaporated under reduced pressure so as to obtain 260 mg of tetraol 6a (yield=80%).

6a: ¹H (300 MHz, CDCl₃) δ 7.51 (d, J=7.5 Hz, 1H), 7.08 (d, J=7.5 Hz, 1H), 4.45 (brs, 2H), 4.13 (brs, 1H), 3.84 (brs, 1H), 2.66 (t, J=7.5 Hz, 2H), 1.65 (m, 2H), 0.90 (t, J=6.9 Hz, 3H);

¹³C (75 MHz, CDCl₃) δ 162.1, 151.2, 139.3, 128.4, 123.5, 71.8, 66.2, 57.5, 53.7, 39.6, 22.9, 13.7;

EIMS (70 eV) m/z (%) 239 (18), 147 (9), 129 (45), 112 (90), 84 (40), 70 (84), 57 (100); IR cm⁻¹ 3330, 2960, 1600, 1035.

### Example 3

### Preparation of the Derivatives 7a and 8a

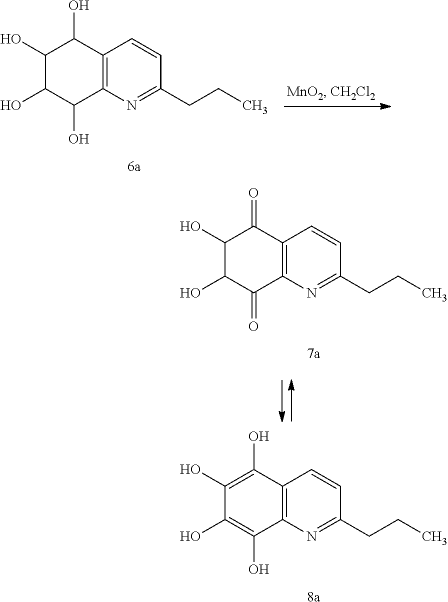

490 mg (5.5 mmol, 25 eq.) of MnO₂ are added portionwise, at ambient temperature, to a 25 ml round-bottomed flask containing 54 mg (0.22 mmol) of tetraol 6a in 3 ml of chloroform. The mixture is stirred for 24 h. The solvent is evaporated off under reduced pressure and the crude residue is purified by silica gel chromatography. The eluent used for separating the products is a CH₂Cl₂/MeOH (9/1, v/v) mixture. The product 7a and 8a (9.8 mg) is isolated and identified by GC-MS, NMR and IR (yield=25%).

7a: ¹H (300 MHz, CDCl₃) δ 8.18 (d, J=8.1 Hz, 1H), 7.35 (d, J=8.1 Hz, 1H), 4.56 (d, J=3.6 Hz, 1H), 4.11 (d, J=3.6 Hz, 1H), 2.87 (t, J=7.8 Hz, 2H), 1.80 (m, 2H), 1.00 (t, J=7.5 Hz, 3H);

¹³C (75 MHz, CDCl₃) δ 190.8, 163.9, 151.4, 137.1, 132.0, 124.2, 71.1, 57.6, 54.0, 40.6, 22.6, 13.8;

EIMS (70 eV) m/z (%) 235 (M, 15), 203 (41), 188 (40), 175 (100), 159 (22), 146 (14), 77 (15);

IR cm⁻¹ 2920, 1710, 1590, 1260, 1115.

### Example 4

### Synthesis of Metabolites of Quinoline 2b

Same procedure as in Example 1.

Mn(TDCPP)Cl and Mn(TDCFP)Cl catalytic oxidation of 2b (500 mg):

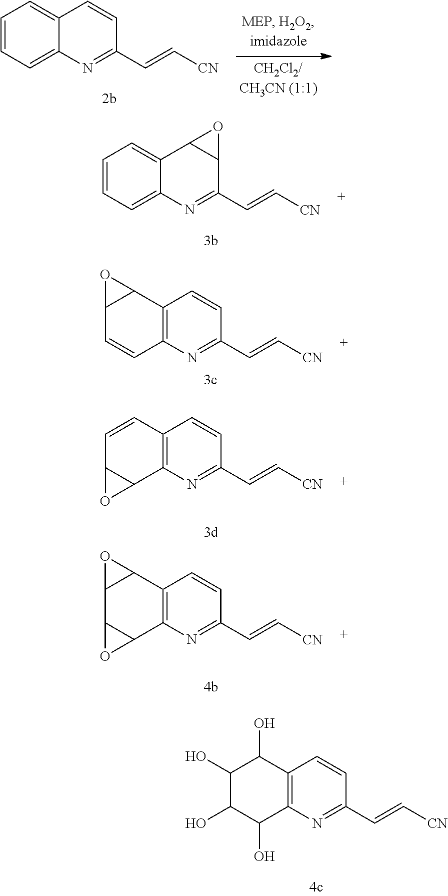

MEP=Mn (TPCFP)Cl: 3b (trace), 3c (3.4%), 3d (1.8%), 4b (34.8%), 4c (2.6%)
MEP=Mn (TDCPP)Cl: 3b (trace), 3c (4.8%), 3d (trace), 4b (17.6%), 4c (7.9%)

### Compound 3b (Trace):

observed only by GC-MS

EIMS (70 eV) m/z (%) 196 (M, 17), 168 (20), 140 (15), 129 (15), 128 (100);

### Compound 3c (18.8 mg):

¹H (300 MHz, CDCl₃) δ 7.95 (d, J=7.8 Hz, 1H), 7.41 (d, J=15.9 Hz, 1H), 7.25 (d, J=7.5 Hz, 1H), 6.94 (dd, J=9.6 Hz; 0.6 Hz, 1H), 6.80 (dd, J=9.9 Hz; 3.9 Hz, 1H), 6.64 (d, J=16.2 Hz, 1H), 4.49 (d, J=3.9 Hz, 1H), 4.17 (d, J=0.9 Hz, 1H);

¹³C (75 MHz, CDCl₃) δ 151.6, 150.9, 148.1, 138.2, 133.1, 131.3, 129.8, 122.5, 117.8, 101.5, 56.1, 53.0;

EIMS (70 eV) m/z (%) 196 (M, 100), 179 (12), 168 (17), 145 (30), 143 (20);

IR cm⁻¹: 2920, 2215, 1565, 1455, 970;

### Compound 3d (10.1 mg):

¹H (300 MHz, CDCl₃) δ 7.62 (d, J=7.8 Hz, 1H), 7.41 (d, J=16.2 Hz, 1H), 7.32 (d, J=7.8 Hz, 1H), 6.75 (d, J=9.6 Hz, 1H), 6.72 (d, J=15.3 Hz, 1H), 6.60 (dd, J=9.6 Hz, 3.6 Hz, 1H), 4.64 (d, J=3.6 Hz, 1H), 4.19 (td, J=3.6 Hz, 1.5 Hz, 1H);

¹³C (75 MHz, CDCl₃) δ 151.5, 149.7, 147.8, 146.0, 136.2, 129.6, 128.8, 128.2, 124.3, 117.9, 101.1, 58.1, 53.7;

EIMS (70 eV) m/z (%) 196 (M, 100), 168 (34), 145 (12), 140 (12), 70 (9), 63 (9);

IR cm⁻¹: 2920, 2853, 2215, 1740, 1465, 1260, 965;

### Compound 4b (205.3 mg):

¹H (300 MHz, CDCl₃) [symbol] 7.76 (d, J=7.6 Hz, 1H), 7.35 (d, J=16.0 Hz, 1H), 7.26 (d, J=7.6 Hz, 1H), 6.67 (d, J=16.0 Hz, 1H), 4.08 (dd, J=11.2, 3.2 Hz, 1H), 3.88 (d, J=4.0 Hz, 1H), 3.73 (d, J=4.0 Hz, 1H)

¹³C (75 MHz, CDCl₃) [symbol] 153.0, 151.0, 147.2, 139.6, 129.4, 124.1, 117.7, 102.0, 55.5, 54.8, 52.9, 50.5;

EIMS (70 eV) m/z (%) 212 (M, 32), 183 (100), 155 (86), 128 (18), 102 (14), 77 (11);

IR cm⁻¹ 3070, 3020, 2920, 2850, 2220, 1730, 1570, 1455, 960;

### Compound 4c (15.2 mg):

¹H (300 MHz, Acetone-d6) δ 7.96 (d, J=7.8 Hz, 1H), 7.66 (d, J=16.2 Hz, 1H), 7.65 (d, J=7.8 Hz, 1H), 6.81 (d, J=16.2 Hz, 1H), 5.14 (m, 1H), 4.74 (m, 1H), 4.10 (dd, J=3.6 Hz, 0.3 Hz, 1H), 394 (m, 1H);

¹³C (75 MHz, Acetone-d6) δ 155.2, 153.1, 149.2, 142.2, 132.8, 126.6, 119.5, 103.2, 69.6, 58.5, 57.2, 54.0;

EIMS (70 eV) m/z (%) 248 (13), 213 (M, 100), 196 (28), 185 (52), 167 (52), 155 (35), 129 (20), 102 (20), 77 (27);

IR cm⁻¹: 2925, 2220, 1745, 1570, 1445, 960.

### REFERENCES

Synthesis of tetraphenylporphyrins under very mild conditions, Jonathan S. Lindsey, Henry C. Hsu and Irwin C. Schreiman Tetrahedron Lett. 1986, 27(41), 4969-4970.

Formulation

A tablet is prepared using the following ingredients: Compound 4a, ethyl acetate, corn starch, microcrystalline cellulose, carnauba wax, titanium dioxide, ethanol, 2-ethoxyethanol, sodium starch glycolate, ammonium hydroxide, hydroxypropylcellulose, hypromellose, shellac, black iron oxide, red iron oxide, polyethylene glycol, propylene glycol and magnesium stearate.

1. A process for producing molecules chosen from those of formulae (IIa), (IIb), (IIc) and (III), this process being characterized in that it comprises at least one step during which a molecule corresponding to general formula (I): 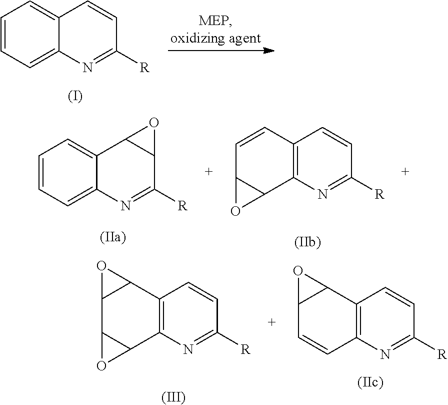
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group;
is reacted with a metalloporphyrin in the presence of an oxidizing agent, in a solvent, so as to give the products (IIa), (IIb), (IIc) and (III).

2. The process as claimed in claim 1, in which R comprises a CH₂ group in the alpha-position with respect to the quinoline ring, this process also comprising the conversion of the molecules of formula (Ia) to molecules of formula (IV) in which R′ represents a group chosen from:
a C₁ to C₁₄ alkyl group, a C₂ to C₁₄ alkenyl group or a C₂ to C₁₄ alkynyl group;
a C₁ to C₁₄ alkyl or C₂ to C₆ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₆ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₄ alkenyl or alkynyl group substituted with at least one C₁ to C₇ trialkylsilyl group:

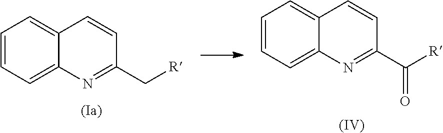

3. The process as claimed in claim 1, in which the metalloporphyrin is chosen from:

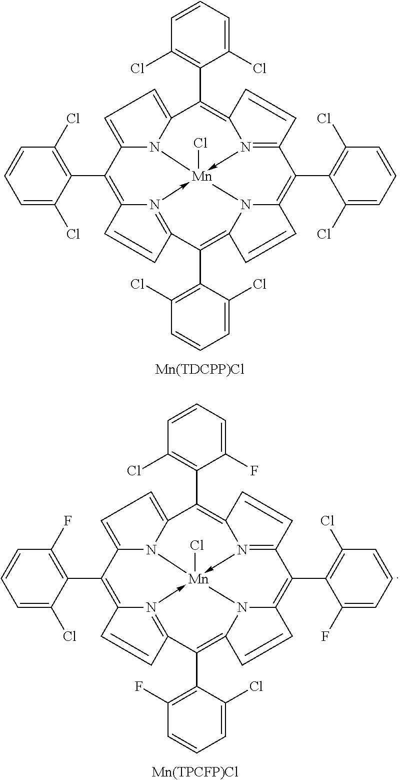

4. The process as claimed in claim 3, in which the oxidizing agent is H₂O₂ and the solvent is a mixture of CH₂Cl₂ and acetonitrile.

5. The process as claimed in claim 1, applied to the n-propyl-2-quinoline 2a so as to give the products 3a, 4a, 5a according to the scheme:

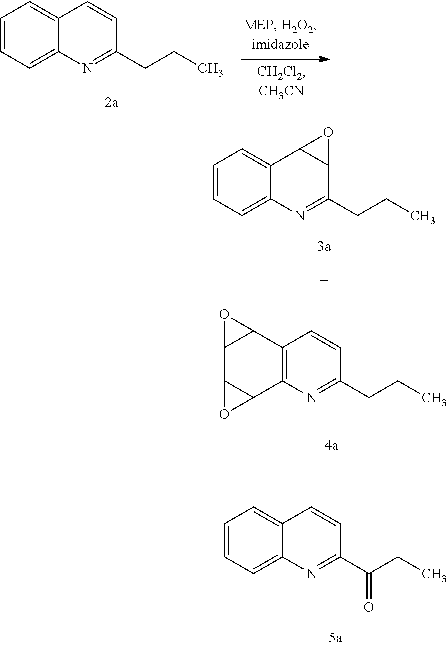

6. The process as claimed in claim 1, applied to the 2-(3-cyano-n-2-propenyl)quinoline 2b, so as to give the products 3b, 3c, 3d, 4b and 4c according to the scheme:

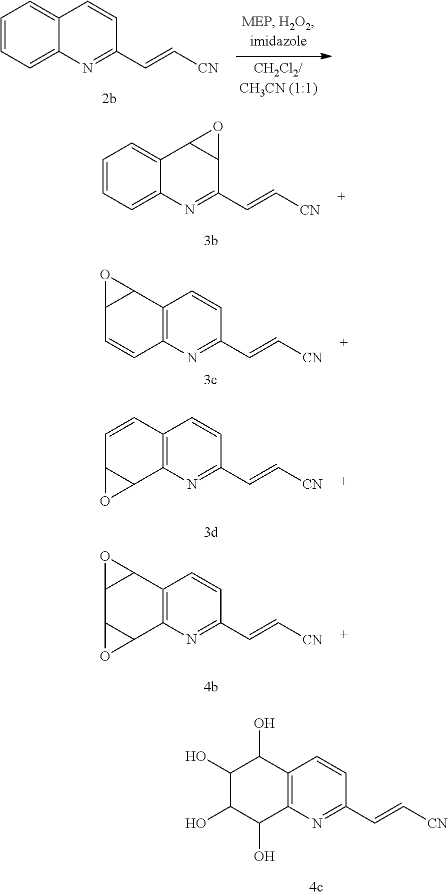

7. A molecule of formula (IIa), which can be obtained by means of a process as claimed in claim 1: 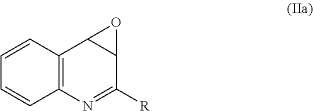
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group;
with the exclusion of the case where R=n-propyl.

8. A molecule of formula (IIb), which can be obtained by means of the process as claimed in claim 1: 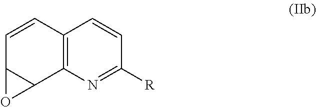
in which R represents a group chosen from:
a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group;
with the exclusion of the case where R=n-propyl.

9. A molecule of formula (IIc), which can be obtained by means of the process as claimed in claim 1: 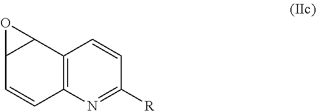
in which R represents a group chosen from:
a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group;
with the exclusion of the case where R=n-propyl.

10. A molecule of formula (III), which can be obtained by means of the process as claimed in claim 1: 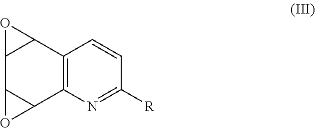
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following ups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group.

11. A molecule of formula (IV), which can be obtained by means of the process as claimed in claim 1:
in which R′ represents a group chosen from:
a C₁ to C₁₄ alkyl group, a C₂ to C₁₄ alkenyl group or a C₂ to C₁₄ alkynyl group;
a C₁ to C₁₄ alkyl or C₂ to C₆ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₆ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₄ alkenyl or alkynyl group substituted with at least one C₁ to C₇ trialkylsilyl group, with the exclusion of the cases where R′ represents a group chosen from:

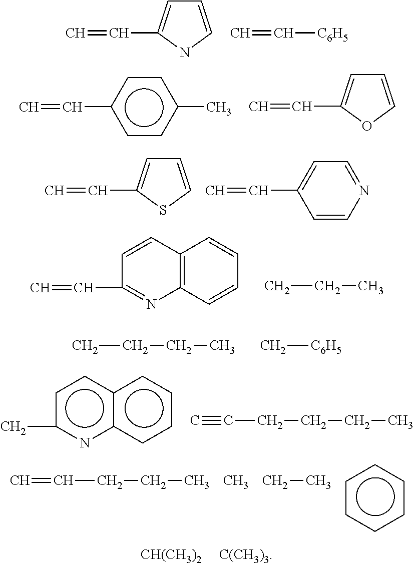

12. A process for producing a molecule corresponding to formula (V), characterized in that it comprises at least one step in which a molecule of formula (III) as claimed in claim 10 is subjected to a treatment of hydrolysis in an acid medium according to the scheme below:

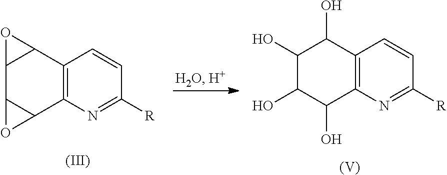

13. A molecule of formula (V), which can be obtained by means of the process as claimed in claim 12: 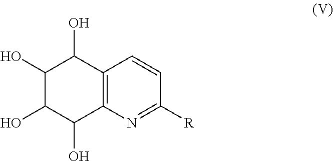
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group.

14. A process for producing a molecule corresponding to formula (VI) or to formula (VII), characterized in that it comprises at least one step in which a molecule of formula (V) as claimed in claim 13 is subjected to an oxidation treatment according to the scheme below:

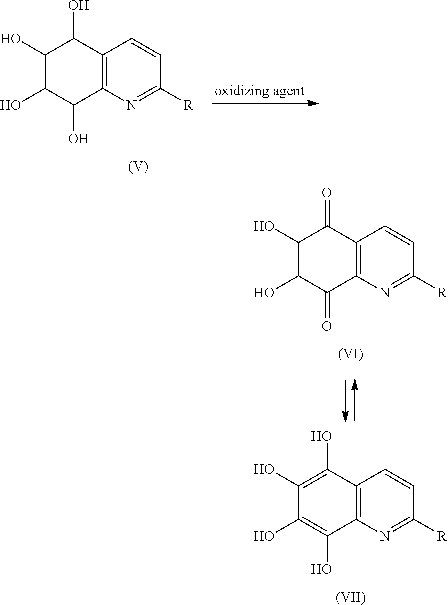

15. The process as claimed in claim 12, applied to the product 4a so as to give the products 6a, 7a and 8a according to the scheme below:

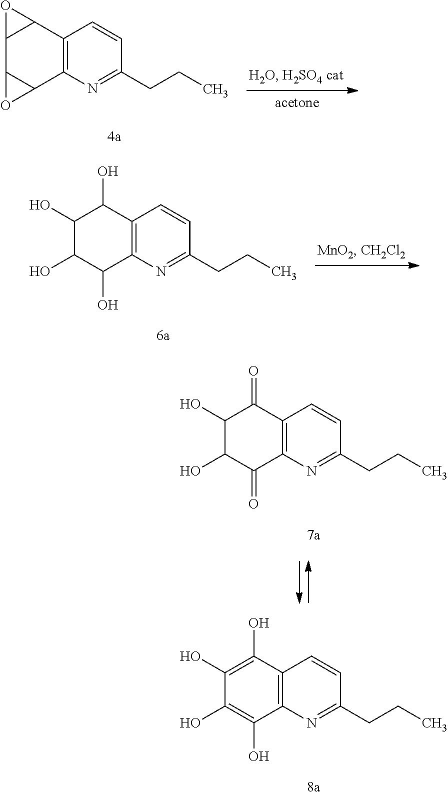

16. A molecule which can be obtained by means of the process as claimed in claim 14, characterized in that it corresponds to formula (VI) or to formula (VII) 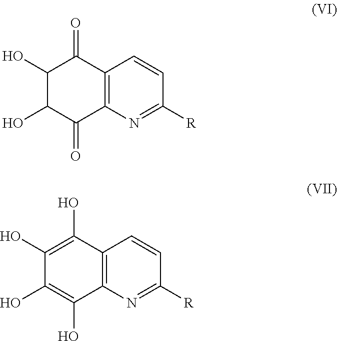
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₁₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group.

17. A molecule which can be obtained by means of the process as claimed in claim 5, chosen from the following list:

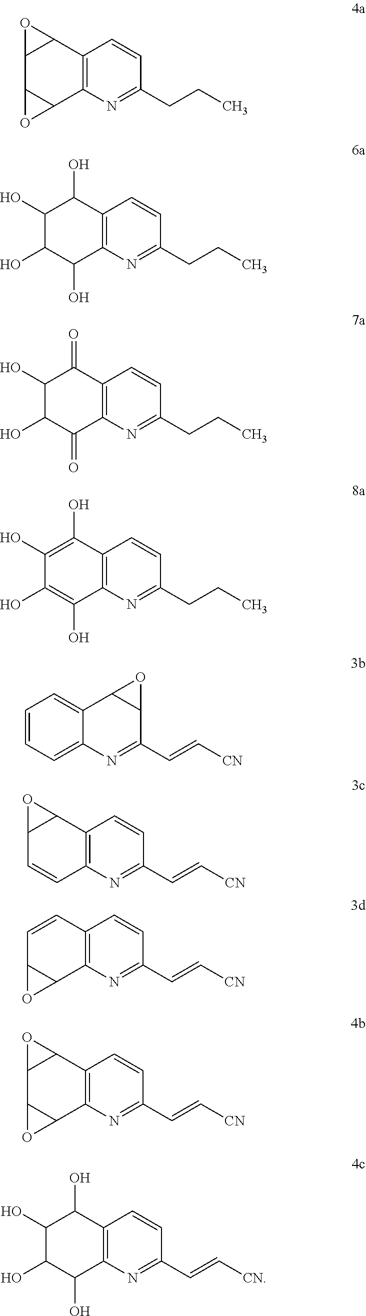

18. A pharmaceutical composition comprising a pharmaceutical carrier and a product of one of the following formula (IIa), (IIb), (IIc), (III), (IV), (V) or (VI) as set forth below:
(IIa): 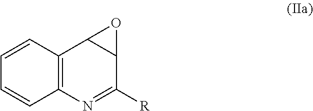
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₁ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₁ to C₁₅ alkenyl or alkynyl group substituted with at least one C₁ to C₇ trialkylsilyl group;
with the exclusion of the case where R=n-propyl;
(IIb): 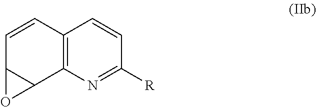
in which R represents a group chosen from:
a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group;
with the exclusion of the case where R=n-propyl;
(IIc): 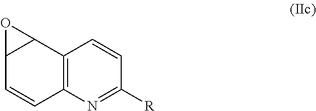
in which R represents a group chosen from:
a C₁ to C₁₅ alkyl group, a C₂ to C₁₅ alkenyl group, a C₁ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group;
with the exclusion of the case where R=n-propyl;
(III): 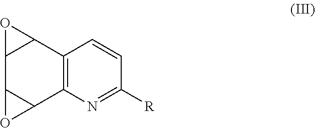
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following ups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group;
(IV): 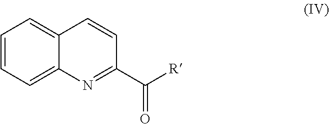
in which R′ represents a group chosen from:
a C₁ to C₁₄ alkyl group, a C₂ to C₁₄ alkenyl group or a C₂ to C₁₄ alkynyl group;
a C₁ to C₁₄ alkyl or C₂ to C₆ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₆ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₄ alkenyl or alkynyl group substituted with at least one C₁ to C₇ trialkylsilyl group, with the exclusion of the cases where R′ represents a group chosen from: 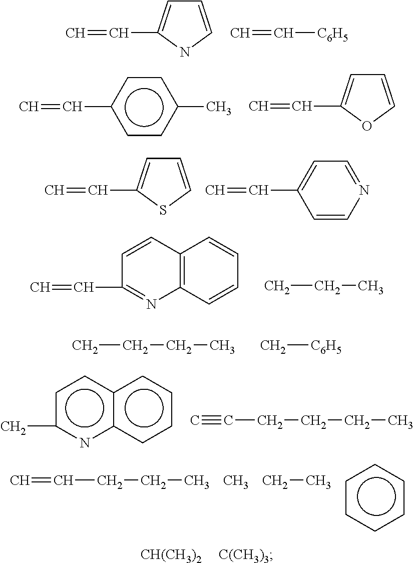
(V): 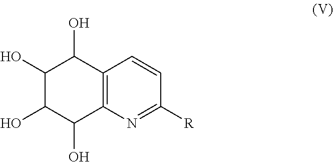
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈ heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group; and
(VI): 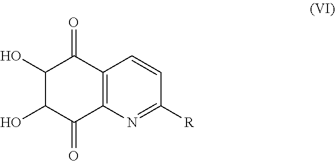
in which R represents a group chosen from:
a hydrogen atom, a C₁ to C₁₅ alkyl group, a C₂ to C₁₅ alkenyl group, a C₂ to C₁₅ alkynyl group, a formyl group or a C₄ to C₁₈ heteroaryl group, the latter being optionally substituted with one or more hydroxyl groups;
a C₁ to C₁₅ alkyl or C₂ to C₇ alkenyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₁ to C₉ alkenyloxycarbonyl, cyano (CN), amine (NH₂), C₁ to C₇ alkoxy, phenoxy, C₃ to C₆ cycloalkyl, C₆ to C₁₂ aryl, C₄ to C₁₈ heteroaryl, C₄ to C₁₈heteroaryloxy, C₆ to C₁₂ arylsulfone, C₁ to C₇ alkylsulfone, C₁ to C₇ thioalkyl and C₁ to C₇ aminoalkyl;
a C₂ to C₇ alkynyl group bearing at least one substituent chosen from oxygen, halogens and the following groups: hydroxyl, formyl, carboxyl, C₇ to C₁₃ aryloxycarbonyl, C₂ to C₈ alkyloxycarbonyl, C₃ to C₉ alkenyloxycarbonyl, cyano, C₆ to C₁₂ aryl, C₄ to C₁₈ heteroaryl, C₆ to C₁₂ arylsulfone, C₁ to C₇ alkylsulfone, C₁ to C₇ thioalkyl and C₁ to C₇ aminoalkyl;
a C₂ to C₁₅ alkenyl or alkynyl group substituted with at least one C₁ to C₇ trialkylsilyl group.

19. The composition as claimed in claim 18, for use in the prevention or treatment of a disease selected from infections caused by protozoa, such as leishmaniasis, trypanosomiasis, toxoplasmosis, and/or infections caused by retroviruses, for instance HIV or HTLV.
